(19) United States
(12) Patent Application Publication
(10) Pub. No.: US 2017/0360568 A1
(43) Pub. Date: Dec. 21, 2017

(54) ORTHOPAEDIC IMPLANT

(71) Applicant: KABUSHIKI KAISHA TOSHIBA, Tokyo (JP)

(72) Inventor: Sema DUMANLI OKTAR, Bristol (GB)

(73) Assignee: KABUSHIKI KAISHA TOSHIBA, Tokyo (JP)

(21) Appl. No.: 15/504,605

(22) PCT Filed: Mar. 3, 2015
(86) PCT No.: PCT/GB2015/050609

§ 371 Date: Feb. 16, 2017

(51) Int. Cl. A61F 2/36; A61F 2/46; A61F 2/30

(52) U.S. Cl. CPC A61F 2/36; A61F 2/4607; A61F 2002/4666; A61F 2002/3067; A61F 2002/4672; A61F 2002/3082

(57) ABSTRACT

An orthopaedic implant comprising: a body portion for replacing a missing joint or bone, or for supporting a joint or bone within a patient's body; a slot antenna for effecting wireless communication between the implant and an external device, wherein the slot antenna comprises a first surface in which is arranged a slot for receiving and/or transmitting wireless communication signals; wherein the slot antenna is comprised within the body portion, such that the slot is exposed to the environment outside of the implant.

### FIELD

Embodiments described herein relate to an orthopaedic implant.

### BACKGROUND

Orthopaedic implants can be equipped with sensors, antenna(s) and microelectronics to form a wireless link for communicating data between the implant and a remote device. The data may include information concerning the condition of the implant and/or the surrounding environment (for example, the tissue in which the implant is located). As an example, the link may be used to report an increase in temperature or pressure in the vicinity of the implant, which could, respectively, indicate an infection or a problem with the original positioning of the implant. The wireless link can also be used to transmit data to the implant, in order to control various functions or cause the implant to adopt a different configuration, for example.

The human body comprises a heavily RF hostile environment; the amplitude of radio waves diminishes quickly due to the lossy nature of the human tissues. In order to maintain the performance of the communication link, the design and configuration of the antenna(s) is critical.

### BRIEF DESCRIPTION OF DRAWINGS

Embodiments of the invention will now be described by way of example with reference to the accompanying drawings in which:

FIG. 1 shows a schematic of a conventional orthopaedic hip implant:

FIG. 2 shows a schematic of an orthopaedic implant according to an embodiment; and

FIG. 3 shows an example of how a slot antenna may be inserted into an orthopaedic implant during a surgical procedure, in accordance with embodiment.

### DETAILED DESCRIPTION

According to a first embodiment, there is provided an orthopaedic implant comprising:
a body portion for replacing a missing joint or bone, or for supporting a joint or bone within a patient's body;
a slot antenna for effecting wireless communication between the implant and an external device, wherein the slot antenna comprises a first surface in which is arranged a slot for receiving and/or transmitting wireless communication signals;
wherein the slot antenna is comprised within the body portion, such that the slot is exposed to the environment outside of the implant.

In some embodiments, the first surface comprises an electrically conductive surface of the body portion.

In some embodiments, the slot antenna is a cavity backed slot antenna. The cavity of the cavity backed slot antenna may be located inside the body portion.

At least one of the walls of the cavity may be formed by a wall of the body portion. In the event that one or more of the walls of the cavity are formed from walls of the body portion, one or more of those walls of the body portion may be electrically conductive.

In some embodiments, the cavity backed slot antenna is insertable into the cavity in the body portion.

In some embodiments, the first surface is a ground plane of the cavity backed slot antenna.

In some embodiments, the first surface comprises part of the outer surface of the implant. The first surface may be shaped so as to match the contours of the outer surface of the body portion.

In some embodiments, the implant includes sensing means for sensing temperature and/or pressure in the environment surrounding the implant and the antenna is configured to transmit data reflecting the temperature and/or pressure to the external device.

According to a second embodiment, there is provided a method for placing a slot antenna in the body of an orthopaedic implant, the body of the orthopaedic implant having a cavity for receiving a broach handle for use in positioning the implant within the patient's body, the method comprising:
removing the broach handle from the body of the implant;
inserting the slot antenna into the cavity vacated by the broach handle, such that the slot is exposed to the environment outside of the implant.

As described herein, an orthopedic implant can be understood to refer to a medical device that is manufactured to replace a missing joint or bone or to support a damaged bone. Such an implant may be provided for different parts of the body, including the hip, knee or shoulder, for example. Typically, such implants are fabricated using stainless steel and titanium alloys. A plastic coating may be provided in order to act as artificial cartilage.

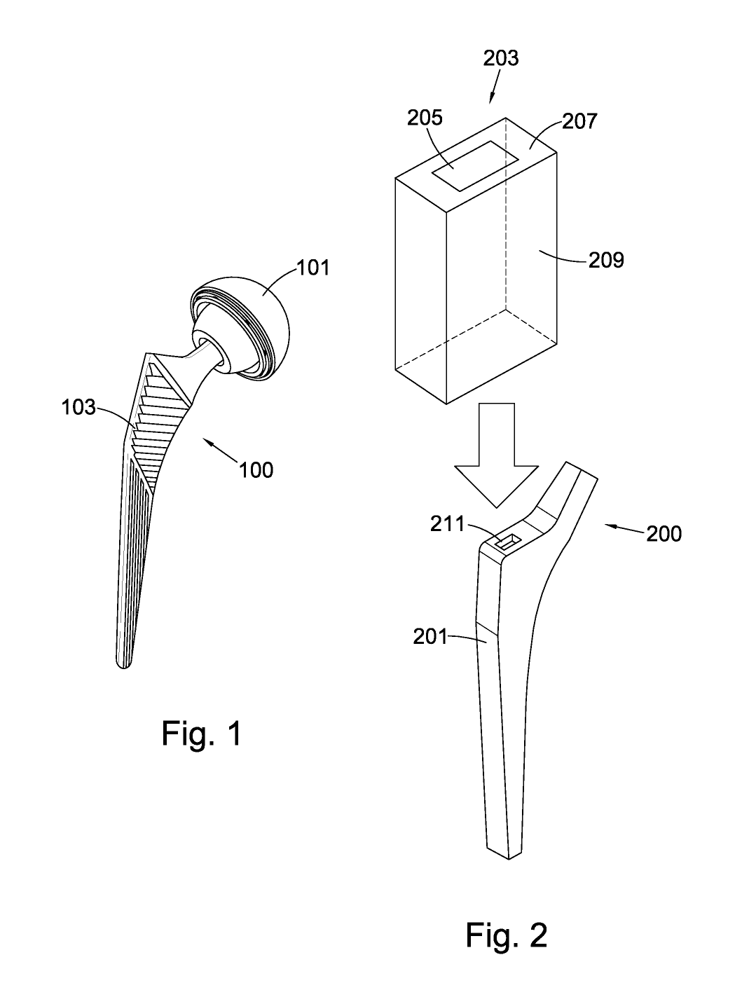

A conventional implant 100 is shown in FIG. 1. In this example, the implant is a hip implant, designed to replace the joint between a patient's pelvis and femur (thigh bone). The implant comprises a head portion 101 and a stem portion 103. The head portion is received in a socket in the pelvis, whilst the stem is inserted into the femur. The stem can rotate relative to the head, allowing the patient to move their leg as normal.

FIG. 2 shows an example of an orthopaedic implant 200 according to an embodiment. In this embodiment, the implant 200 is a hip implant similar to FIG. 1, but it will be appreciated that the implant could be one of any types of orthopaedic implant used for different parts of a patient's body. The implant 200 includes a body portion 201, which provides the main structural component of the implant, and which performs the same role as the conventional implant shown in FIG. 1. The implant 200 further comprises a slot antenna 203 that is used to provide a wireless communications link between the implant and an external device. In the present embodiment, the slot antenna is received within the body portion 201, as shown by the large arrow. When received within the body portion, the slot 203 is exposed to the exterior of the implant.

The external device may be one of any known communication devices having a wireless receiver and/or transmitter, including for example a laptop computer, mobile telephone or tablet. The external device may be used by a physician to monitor the state of the patient's implant following surgery to set the implant in place. The antenna can be used to transmit information about the implant's environment once the implant has been positioned within the patient's body, and/or to receive information or instructions to reconfigure the implant (for example, to cause the head portion of the implant to adopt a different position relative to the stem portion).

In use, the power dissipated by the antenna can be calculated by integrating the ohmic losses in the near field, these losses being caused by the conductivity of the tissues. In this respect, a slot antenna (which is an example of a magnetic antenna) provides an advantage over electric antennas, in that the electric field in the near-field of the antenna is smaller. Consequently, the slot antenna will induce fewer currents in the surrounding tissue, thereby reducing the ohmic losses. In addition, since the slot antenna is exposed to the outside of the implant, the wireless communication signals will not be blocked by the body of the implant. Accordingly, the integrity of the wireless communication link can be maintained without sacrificing the physical strength or the size and the shape of the orthopaedic implant.

In the embodiment shown in FIG. 2, the slot antenna 203 is a cavity backed slot antenna. The antenna comprises a slot 205 located in a first surface 207, which is backed by a cavity 209. It will be understood that the first surface 207, together with the slot 205 comprises the radiating element that actually translates guided waves into free space waves, whilst the cavity 209 (which is formed by the first surface 207 and the other 5 walls of the unit shown in FIG. 2) provides a means for directing the free space waves in a particular direction.

In the present embodiment, the cavity backed slot antenna 203 is received within a cavity 211 in the body portion. When the cavity backed slot antenna is received within the body portion, the cavity 209 extends from the surface 207 towards the inside of the body portion.

Implementing a cavity backed slot antenna may help to improve the strength of the wireless signal received from the antenna, as most of the radiation from the antenna will be transmitted in a single direction.

It will be understood that in some embodiments, the walls of the cavity 211 in the body portion may themselves provide the cavity for the cavity-backed slot antenna 203; that is, it may not be necessary to provide a separate walled cavity 209 for the antenna, but instead this cavity can be fashioned from the body portion itself. In such a case, the slot antenna may be provided as an additional piece of metal (for example, a planar sheet) that is placed at one end of the cavity 211 in the body portion.

In the example shown in FIG. 2, the antenna is positioned so as to face upwardly from the body portion. However, it will be understood that the location of the slot antenna within the body of the implant can be tailored to both the shape of the implant and its intended position within the body of the patient.

In some embodiments, the surface in which the slot is located may itself comprise a conducting (outer) surface of the implant; that is, the outside surface of the implant may form a wall of the cavity backing the slot antenna, or the ground plane of the slot antenna.

In some embodiments, the orthopaedic implant may be provided as a single unit, in which the slot antenna is incorporated (fixed) within the body portion; alternatively, the antenna may be provided as a separate component from the body of the implant, which can then be inserted into the body portion during surgery.

When positioned in the body portion, the slot antenna can be connected to other circuitry that is housed within the implant and which is used to monitor the implant's environment, for example. Such circuitry may include sensors for performing measurements of temperature and/or pressure, as well as standard components for converting those measurements into signals capable of being transmitted by slot antenna and/or for processing of signals being received by the antenna.

In some embodiments, the surface in which the slot is located is a flat surface. In some embodiments, the surface may be shaped so as match the contours of the body portion; that is, there may be a smooth transition between the first surface and the rest of the outer surface of the body portion. The surface in which the slot is located may itself comprise an outer surface of the implant, or be substantially flush with the outer surface of the implant. The antenna can, therefore, be positioned within the body portion without protruding from the structure and adversely affecting the mechanical strength.

In some embodiments, the slot antenna is configured to transmit at frequencies in one or more of the 2.4 GHz ISM band, 5.2 GHz ISM Band and Lower UWB (for example, in a frequency range from 3.1 GHz to 4.8 GHz).

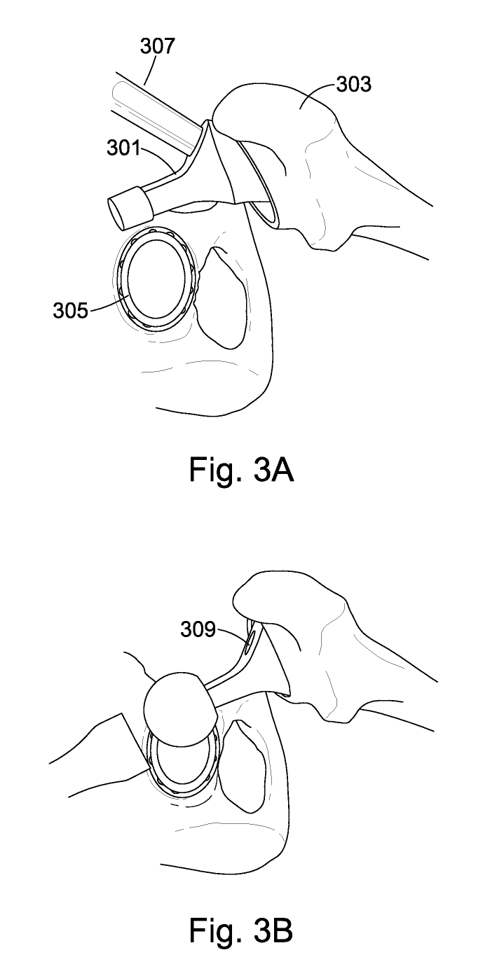

FIG. 3 shows an example of how a slot antenna may be placed in an orthopaedic hip implant according to an embodiment, as part of a surgical procedure. FIG. 3A shows a body portion 301 of the implant being brought into position inside the patient's femur 303, before the head of the implant is set within the socket 305 in the pelvis. The implant is manoeuvred using a broach handle 307, one end of which is inserted into a cavity located within the body portion. FIG. 3B shows the implant with the broach handle removed. At this point, the cavity 309 in which the broach handle was inserted has now been vacated and is ready to receive the slot antenna. The slot antenna can be inserted into the cavity as described above in relation to FIG. 2. In this way, the cavity in the body portion can serve a dual purpose of assisting the positioning of the implant during surgery, and thereafter providing a cavity for housing a cavity-backed antenna or even forming part of the cavity backed antenna itself.

While certain embodiments have been described, these embodiments have been presented by way of example only and are not intended to limit the scope of the invention. Indeed, the novel methods, devices and systems described herein may be embodied in a variety of forms; furthermore, various omissions, substitutions and changes in the form of the methods and systems described herein may be made without departing from the spirit of the invention. The accompanying claims and their equivalents are intended to cover such forms or modifications as would fall within the scope and spirit of the inventions.

1. An orthopaedic implant comprising:
a body portion for replacing a missing joint or bone, or for supporting a joint or bone within a patient's body;
a slot antenna for effecting wireless communication between the implant and an external device, wherein the slot antenna comprises a first surface in which is arranged a slot for receiving and/or transmitting wireless communication signals;
wherein the slot antenna is comprised within the body portion, such that the slot is exposed to the environment outside of the implant.

2. An orthopaedic implant in which the first surface comprises an electrically conductive surface of the body portion.

3. An orthopaedic implant according to claim 1, wherein the slot antenna is a cavity backed slot antenna.

4. An orthopaedic implant according to claim 3, wherein the cavity of the cavity backed slot antenna is located inside the body portion.

5. An orthopaedic implant according to claim 4, wherein at least one of the walls of the cavity is formed by a wall of the body portion.

6. An orthopaedic implant according to claim 5, wherein said wall(s) of the body portion are electrically conductive.

7. An orthopaedic implant according to claim 4, wherein the cavity backed slot antenna is insertable into the cavity in the body portion.

8. An orthopaedic implant according to claim 2, wherein the first surface is a ground plane of the cavity backed slot antenna.

9. An orthopaedic implant according to claim 1, wherein the first surface comprises part of the outer surface of the implant.

10. An orthopaedic implant according to claim 9, wherein the first surface is shaped so as to match the contours of the outer surface of the body portion.

11. An orthopaedic implant according to claim 1, wherein the implant includes sensing means for sensing temperature and/or pressure in the environment surrounding the implant and the antenna is configured to transmit data reflecting the temperature and/or pressure to the external device.

12. A method for placing a slot antenna in the body of an orthopaedic implant, the body of the orthopaedic implant having a cavity for receiving a broach handle for use in positioning the implant within the patient's body, the method comprising:
removing the broach handle from the body of the implant;
inserting the slot antenna into the cavity vacated by the broach handle, such that the slot is exposed to the environment outside of the implant.
